(19) United States
(12) Patent Application Publication
(10) Pub. No.: US 2009/0299100 A1
(43) Pub. Date: Dec. 3, 2009

(54) Fossil Fuel Desulfurization

(75) Inventors: Tong Ren, West Lafayette, IN (US); Julia E. Barker Paredes, Miami, FL (US)

Correspondence Address: VENABLE LLP, P.O. BOX 34385, WASHINGTON, DC, 20043-9998, US

(73) Assignee: UNIVERSITY OF MIAMI, Miami, FL (US)

(21) Appl. No.: 11/794,747

(22) PCT Filed: Dec. 23, 2005
(86) PCT No.: PCT/US05/46909

§ 371 Date: Nov. 20, 2008

Related U.S. Application Data
(60) Provisional application No. 60/641,436, filed on Jan. 6, 2005.

(51) Int. Cl. C07C 315/02; C09K 3/00

(52) U.S. Cl. 568/28; 252/186.1; 252/186.43

(57) ABSTRACT

A method for oxidizing an organic sulfide by combining an alkali borate, a solvent, hydrogen peroxide, and the organic sulfide, and allowing the alkali borate, the hydrogen peroxide, and the organic sulfide to interact to produce an oxidized organic sulfide.

The invention described and claimed herein was made in part with funds from Grant No. DAAD 190110708 from the Army Research Office. The U.S. Government has certain rights in the invention.

### BACKGROUND OF THE INVENTION

1. Field of the Invention

This invention relates to a method of desulfurizing organic compounds. Specifically, the invention relates to a method of oxidizing organic sulfides, and extracting resultant organic sulfones into a solvent.

2. Description of the Related Art

Commonly occurring organic sulfur compounds in fossil fuels, that is, in hydrocarbon fuels that are derived from substances extracted from the earth, such as oil, coal, and natural gas, include mercaptanes, alkyl sulfides, alkyl disulfides, aryl sulfides, aryl disulfides, thiophene, benzothiophene (BT), and dibenzothiophene (DBT).¹ When fossil fuels are burned, organic sulfur compounds are converted to sulfur oxides, such as sulfur dioxide and sulfur trioxide, which are the major culprits of acid rain. An approach to satisfying environmental regulations is desulfurization. Current desulfurization technology is mainly based on hydrotreating or hydrodesulfurization processes, which typically reduce the sulfur level in fuel to 300-500 (weight) ppm.² Such a range of sulfur content will soon become unacceptable under recently established stringent regulations on the sulfur content in transportation fuels. For instance, the sulfur content limits in gasoline and diesel fuel have been set as 30-50 ppm in Europe and the United States starting in 2005; further reduction of these limits is expected in coming years.^{1,3-5} Hydrodesulfurization processes (HDS) are often viewed as mature technologies,¹ and it has been pointed out that the challenge posed by new ultra-low sulfur regulations cannot be met through the incremental improvement of HDS processes.⁵ Polyaromatic sulfur containing compounds, such as benzothiophene (BT), dibenzothiophene (DBT), and alkyl substituted derivatives of BT and DBT, are difficult to hydrogenate and can limit the ability of HDS processes to reduce the sulfur content of fuels.^{2,6} FIG. 1 presents a gas chromatogram, obtained from a gas chromatograph with atomic emission detection, indicating the presence of a number of different polyaromatic sulfur containing compounds in light oil.⁹

Conversion/extraction desulfurization (CED) is a leading candidate among deep desulfurization technologies.^{1,7} CED technology can be based on converting organic sulfur compounds to sulfones and subsequently removing sulfones by liquid-liquid extraction. For example, the laboratory of Dr. Tetsuo Aida has demonstrated that peroxycarboxylic acids, that is, acids produced from mixtures of a carboxylic acid and hydrogen peroxide, readily convert DBT and its alkyl derivatives into corresponding sulfones.⁷ Specifically, Aida et al. note that 4,6-dimethyl dibenzothiophene, one of the most difficult compounds to remove from fuel with HDS because of the steric hindrance of 4,6-dimethyl substituents, undergoes faster conversion than DBT in a mixture of formic acid and hydrogen peroxide. However, based on the limited amount of information available in open literature, this peroxy acid process appears to require harsh conditions of concentrated acid and elevated temperature.

A desulfurization method should preferably use materials of low cost. For example, a desulfurization method used for the large scale removal of organic sulfur compounds from fossil fuels should use low cost materials if possible, given the commodity nature of fossil fuel markets.

In short, there is a need for a method for reducing the sulfur content of fossil fuel to a low concentration, which can be performed with low cost materials, and can be performed under mild, for example, ambient, temperature conditions, without the use of concentrated acid.

### SUMMARY OF THE INVENTION

It is therefore an object of the present invention to provide novel methods for reducing the sulfur content of fossil fuel to a low concentration, which can be performed with low cost materials, and can be performed under mild, for example, ambient, temperature conditions, without the use of concentrated acid. This international application claims benefit to the U.S. Provisional Application Ser. No. 60/641,436, which is hereby incorporated by reference.

In one embodiment, the invention provides a method for oxidizing an organic sulfide, comprising combining an alkali borate, the organic sulfide, and a solvent; and allowing the alkali borate and the organic sulfide to interact to produce an oxidized organic sulfide, for example, an organic sulfone. The solvent is preferably a polar solvent, for example, water, alcohol, methanol, ethanol, 1-propanol, 2-propanol, 1-butanol, 2-butanol, 2-methyl-1-propanol, 2-methyl-2-propanol, acetone, N,N′-dimethylformamide, or acetonitrile, or mixtures thereof. In one preferred embodiment, the solvent comprises a mixture of acetonitrile and water. Mixtures of acetonitrile and water having a volumetric ratio of from about 1:10 to about 10:1 are particularly suitable, particularly mixtures having a volumetric ratio of from about 1:3 to about 3:1, and most particularly those having a ratio of 1:1.

As the alkali borate, lithium borate, sodium borate, and potassium borate are particularly suitable. The alkali borate may be an alkali perborate, e.g., sodium perborate. An alkali tetraborate, e.g., sodium tetraborate, may be used in conjunction with hydrogen peroxide.

The organic sulfide may be, for example, a mercaptane, an alkyl sulfide, an alkyl disulfide, an aryl sulfide, an aryl disulfide, a monoaromatic sulfur-containing compound, thiophene, a polyaromatic sulfur-containing compound, benzothiophene, or dibenzothiophene. Methyl phenyl sulfide, ethyl phenyl sulfide, diphenyl sulfide, and dibenzothiophene are particularly suitable.

In another embodiment, the invention provides a method for oxidizing an organic sulfide, comprising combining an alkali borate, the organic sulfide, hydrogen peroxide, and a solvent, and allowing the alkali borate, hydrogen peroxide, and the organic sulfide to interact to produce an oxidized organic sulfide, for example, an organic sulfone. The solvent is preferably a polar solvent, for example, water, alcohol, methanol, ethanol, 1-propanol, 2-propanol, 1-butanol, 2-butanol, 2-methyl-1-propanol, 2-methyl-2-propanol, acetone, N,N-dimethylformamide, or acetonitrile, or mixtures thereof. In one preferred embodiment, the solvent comprises a mixture of acetonitrile and water. Mixtures of acetonitrile and water having a volumetric ratio of from about 1:10 to about 10:1 are particularly suitable, particularly mixtures having a volumetric ratio of from about 1:3 to about 3:1, and most particularly those having a ratio of 1:1.

As the alkali borate, lithium borate, sodium borate, and potassium borate are particularly suitable. The alkali borate may also be an alkali tetraborate or perborate (e.g., sodium tetraborate or sodium perborate). The molar ratio of alkali borate to organic sulfide is generally less than or equal to about 20 mol %, and can be less than or equal to about 5 mol %.

The organic sulfide may be, for example, a mercaptane, an alkyl sulfide, an alkyl disulfide, an aryl sulfide, an aryl disulfide, a monoaromatic sulfur containing compound, thiophene, a polyaromatic sulfur containing compound, benzothiophene, or dibenzothiophene. Methyl phenyl sulfide, ethyl phenyl sulfide, diphenyl sulfide, and dibenzothiophene are particularly suitable.

The alkali borate can be mixed with the solvent, with or without hydrogen peroxide, to form a borate solution, and the organic sulfide placed in contact with, but not mixed with the borate solution. Alternatively, the alkali borate can be mixed with the solvent, with or without hydrogen peroxide, and with the organic sulfide.

In each of the above-mentioned methods, the organic sulfide may be contained in a hydrocarbon substance (e.g., a fossil fuel, for example, as a contaminant). Accordingly, the invention provides a method for oxidizing an organic sulfide, wherein a hydrocarbon substance comprises the organic sulfide. In this method, the alkali borate, hydrocarbon substance, and solvent are combined; thereby allowing the alkali borate and organic sulfide to interact to produce an oxidized organic sulfide. The invention also provides a method for oxidizing an organic sulfide, wherein the alkali borate, hydrogen peroxide, hydrocarbon substance, and solvent are combined; thereby allowing the alkali borate, hydrogen peroxide, and organic sulfide to interact to produce an oxidized organic sulfide. In these methods, the solvent is preferably a polar solvent. The hydrocarbon substance, solvent, and other components can be mixed to form a mixture; the mixture can be allowed to separate into a polar layer, including the oxidized organic sulfide, and a nonpolar layer, including the hydrocarbon substance. The polar layer can also include the alkali borate and the solvent. The nonpolar layer can then be removed from the polar layer, thus removing the (oxidized) organic sulfide from the hydrocarbon substance; in this manner, a purified hydrocarbon substance can be obtained.

Alternatively, the solvent and other components can be mixed to form a solvent layer, and the solvent layer can be placed into contact but not mixed with the hydrocarbon substance. The hydrocarbon substance can then be separated from the solvent layer, the solvent layer comprising the oxidized organic sulfide.

In an embodiment of the invention, a composition for oxidizing an organic sulfide includes an alkali borate, the organic sulfide, and a solvent. The alkali borate can be an alkali perborate, and the solvent can be a polar solvent. The composition can further include hydrogen peroxide. The alkali borate can be an alkali tetraborate.

### BRIEF DESCRIPTION OF THE DRAWINGS

FIG. 1 includes a gas chromatogram of a light oil detected by atomic emission.

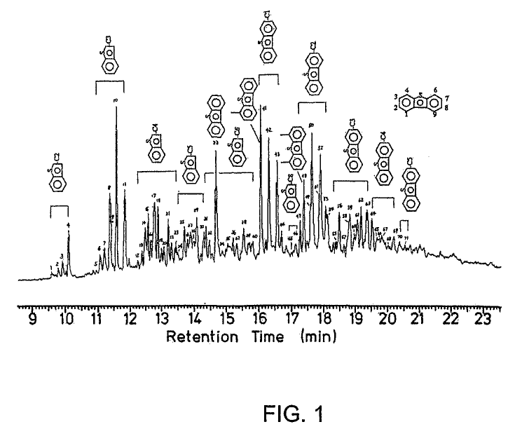

FIG. 2 includes a graph of the absorbance of 290 nm light as a function of time by solutions of methyl phenyl sulfide, sodium perborate, acetonitrile, and water.

FIG. 3 includes a graph of the absorbance of 290 nm light as a function of time by solutions of methyl phenyl sulfide, sodium tetraborate, hydrogen peroxide, acetonitrile, and water.

FIG. 4 includes a graph of the absorbance of 290 nm light as a function of time by solutions of methyl phenyl sulfide, sodium perborate, hydrogen peroxide, acetonitrile, and water.

FIG. 5 includes a graph of the percentages of dibenzothiophene (the sulfide) and of dibenzothiophene sulfone of the total dibenzothiophene derivative product as a function of time for solutions including sodium tetraborate and for solutions including sodium perborate.

### DETAILED DESCRIPTION OF THE INVENTION

In describing preferred embodiments of the present invention, specific terminology is employed for the sake of clarity. However, the invention is not intended to be limited to the specific terminology so selected. All references cited herein are incorporated by reference as if each had been individually incorporated.

In a method according to the present invention for oxidizing an organic sulfide, an alkali borate, the organic sulfide, and a solvent are combined. In this text, the term “alkali borate” refers to all alkali borates, and not just to an alkali tetraborate; for example, the term “sodium borate” encompasses both sodium perborate and sodium tetraborate. The alkali borate and the organic sulfide can be allowed to interact to produce an oxidized organic sulfide. Combining refers to placing ingredients in contact with each other. Combining can include mixing ingredients, for example, mixing the alkali borate, organic sulfide, and solvent with each other through, e.g., mechanical agitation or ultrasound. Alternatively, combining can include placing ingredients into contact with each other without mixing. Combining can include mixing some ingredients and placing another ingredient into contact with the mixed ingredients without mixing. For example, the alkali borate can be dissolved in the solvent to form a borate solution. This borate solution can then be placed into contact with the organic sulfide without mixing; interaction between the ingredients of the borate solution and the organic sulfide can occur across the interface between the borate solution and the organic sulfide. For example, the borate solution can be substantially polar and the organic sulfide substantially nonpolar, so that they form two distinct layers.

Interaction between two or more compounds refers to a process in which one or more of the compounds are either temporarily or permanently changed or transformed. For example, an alkali perborate and an organic sulfide can interact in that they react to oxidize the organic sulfide to, for example, an organic sulfoxide or an organic sulfone. Alternatively, an alkali borate, such as an alkali tetraborate or alkali perborate, an organic sulfide, and another compound can interact in that the alkali borate catalyzes a reaction between the organic sulfide and the other compound. For example, an alkali borate can catalyze a reaction between the organic sulfide and hydrogen peroxide, so that the hydrogen peroxide oxidizes the organic sulfide.

The organic sulfide can include, for example, a mercaptane, an alkyl sulfide, an alkyl disulfide, an aryl sulfide, methyl phenyl sulfide, ethyl phenyl sulfide, diphenyl sulfide, an aryl disulfide, thiophene, a monoaromatic sulfur containing compound, benzothiophene, a polyaromatic sulfur containing compound, or dibenzothiophene or any combination of these.

An oxidized organic sulfide can include, for example, an organic sulfoxide or an organic sulfone. The solvent can include a polar solvent or a polar compound. For example, the solvent can include one or more polar compounds such as water, an alcohol, methanol, ethanol, 1-propanol, 2-propanol, 1-butanol, 2-butanol, 2-methyl-1-propanol, 2-methyl-2-propanol, acetone, N,N′-dimethylformamide, and acetonitrile, or mixtures thereof. For example, the solvent can include a mixture of acetonitrile and water. The solvent can include acetonitrile and water in a volumetric ratio in the range of from about 1:10 to about 10:1. That is, a solvent can be formed by mixing 1 mL of acetonitrile with 10 mL of water, by mixing 10 mL of acetonitrile with 1 mL of water, or by mixing acetonitrile and water in any intermediate proportion. For example, the solvent can include acetonitrile and water in a volumetric ratio in the range of from about 1:3 to about 3:1, or in a volumetric ratio of about 1:1.

The alkali atom in the alkali borate can be selected from any atom in group IA of the periodic table including lithium, sodium, potassium, rubidium, or cesium. For example, a lithium borate, a sodium borate, or a potassium borate can be used.

In a method according to the present invention, an alkali perborate, e.g., sodium perborate, an organic sulfide, and a solvent are combined. The alkali perborate is allowed to react with the organic sulfide to form an organic sulfoxide and/or an organic sulfone. It is thought that the reaction can proceed substantially to completion such that 2 moles of alkali perborate can react with 1 mole of organic sulfide to produce 1 mole of organic sulfone. The reaction can be carried out at room temperature and atmospheric pressure, or other suitable temperatures and pressures as will be known to those of skill in the art.

In another method according to the present invention, an alkali tetraborate, e.g., sodium tetraborate, an organic sulfide, a solvent, and hydrogen peroxide are combined. The alkali tetraborate is thought to function as a catalyst, because 5 mol % of an alkali tetraborate with respect to the organic sulfide can act to have the hydrogen peroxide react with the organic sulfide to convert substantially all of the organic sulfide to organic sulfone; however, the inventors are not bound by this or any other theory as to the mechanisms of the methods disclosed herein. The reaction can be carried out at room temperature and atmospheric pressure, or other suitable temperatures and pressures as will be known to those of skill in the art. An alkali perborate, e.g., sodium perborate, an organic sulfide, a solvent, and hydrogen peroxide can be combined. The alkali perborate is thought to function as a catalyst with this combination of ingredients, because 20 mol % of an alkali perborate with respect to the organic sulfide can act to have the hydrogen peroxide react with the organic sulfide to convert substantially all of the organic sulfide to organic sulfone. The reaction can be carried out at room temperature and atmospheric pressure, or other suitable temperatures and pressures as will be known to those of skill in the art.

For example, sodium tetraborate, dibenzothiophene, a solvent of acetonitrile and water in a 1:1 volumetric ratio, and hydrogen peroxide can be combined; 40 moles of hydrogen peroxide can be used per mole of dibenzothiophene. The sodium tetraborate is thought to function as a catalyst, because 2.5 mol % of sodium tetraborate with respect to the dibenzothiophene can act to have the hydrogen peroxide react with the dibenzothiophene to convert substantially all of the dibenzothiophene to dibenzothiophene sulfone within about one and one-half hours, for example, within about ninety minutes. The reaction product can include dibenzothiophene sulfone with no substantial amounts of dibenzothiophene (the sulfide) or dibenzothiophene sulfoxide. The reaction can be carried out at room temperature and atmospheric pressure, or other suitable temperatures and pressures as will be known to those of skill in the art.

As another example, sodium perborate, dibenzothiophene, a solvent of acetonitrile and water in a 1:1 volumetric ratio, and hydrogen peroxide can be combined; 40 moles of hydrogen peroxide can be used per mole of dibenzothiophene. The sodium perborate is thought to function as a catalyst, because 10 mol % of sodium perborate with respect to the dibenzothiophene can act to have the hydrogen peroxide react with the dibenzothiophene to convert substantially all of the dibenzothiophene (the sulfide) to dibenzothiophene sulfone within about one hour, for example, within about seventy minutes. The reaction product can include dibenzothiophene sulfone with no substantial amounts of dibenzothiophene (the sulfide) or dibenzothiophene sulfoxide. The reaction can be carried out at room temperature and atmospheric pressure, or other suitable temperatures and pressures as will be known to those of skill in the art.

Use of sodium tetraborate or sodium perborate in the oxidization of organic sulfides can be advantageous because sodium tetraborate, also known as borax, and sodium perborate are inexpensive commodity chemicals and do not require special equipment for handling or processing.

A hydrocarbon substance can initially include an organic sulfide; the hydrocarbon substance can also include non-sulfur-containing hydrocarbons. For example, the hydrocarbon substance can be a hydrocarbon fuel, such as a fossil fuel. Thus, a method according to the present invention can be used to remove organic sulfides from a hydrocarbon substance. A hydrocarbon substance including an organic sulfide can be combined with an alkali borate, e.g., sodium perborate, and a solvent. For example, the solvent can be a polar solvent; and the hydrocarbon substance, alkali borate, and polar solvent can be combined through mixing, and allowed to interact. The interaction can be carried out at room temperature and/or atmospheric pressure, or at other suitable temperatures and pressures as known to those of skill in the art. During the interaction, the organic sulfide in the hydrocarbon substance can be oxidized; for example, the organic sulfide can be converted to an organic sulfoxide or an organic sulfone. The oxidized organic sulfide, e.g., an organic sulfone, can be substantially polar and can migrate from a hydrocarbon substance phase to a solvent, e.g., a polar solvent, phase. After a sufficient time, the hydrocarbon substance can have substantially no organic sulfide, or can have a concentration of organic sulfide greatly reduced from the concentration of organic sulfide in the hydrocarbon substance before combination with the alkali borate and the solvent. A sufficient time for interaction can be determined, for example, by monitoring the concentration of the organic sulfide in the hydrocarbon substance, e.g., by monitoring the concentration of the organic sulfide through a gas chromatography-mass spectroscopy technique. The hydrocarbon substance can be separated from the alkali borate, solvent, and oxidized organic sulfide. For example, the hydrocarbon substance can be separated by allowing a polar solvent, the alkali borate, and the oxidized organic sulfide to separate into a polar layer, and by allowing the hydrocarbon substance to separate into a nonpolar layer. The nonpolar layer including the hydrocarbon substance can be removed. The separated, purified hydrocarbon substance can be used or further processed. Thus, a method according to the present invention can be used as a conversion/extraction desulfurization technology.

In another method, the hydrocarbon substance including an organic sulfide is combined with an alkali borate, e.g., sodium tetraborate or sodium perborate, a solvent, and hydrogen peroxide. For example, the solvent can be a polar solvent; and the hydrocarbon substance, alkali borate, solvent, and hydrogen peroxide can be combined through mixing. The combination of the hydrocarbon substance, alkali borate, hydrogen peroxide, and solvent can be allowed to interact. For example, the interaction can be allowed to proceed at room temperature and/or atmospheric pressure, or other suitable temperatures and pressures as will be known to those of skill in the art. After a sufficient time, the hydrocarbon substance can have substantially no organic sulfide, or can have a concentration of organic sulfide greatly reduced from the concentration of organic sulfide in the hydrocarbon substance before combination with the alkali borate and the solvent A sufficient time for interaction can be determined, for example, by monitoring the concentration of the organic sulfide in the hydrocarbon substance, e.g., by monitoring the concentration of the organic sulfide through a gas chromatography-mass spectroscopy technique. The hydrocarbon substance can be separated from the alkali borate, solvent, unreacted hydrogen peroxide, and oxidized organic sulfide. For example, the hydrocarbon substance can be separated by allowing a polar solvent, the alkali borate, unreacted hydrogen peroxide, and the oxidized organic sulfide to separate into a polar layer, and by allowing the hydrocarbon substance to separate into a nonpolar layer. The nonpolar layer including the purified hydrocarbon substance can be removed. The separated, purified hydrocarbon substance can be used or further processed.

In an alternative method, an alkali borate, e.g., sodium perborate, is combined with a solvent, e.g., a polar solvent, to form a borate solution. The borate solution can be contacted, without being mixed, with a hydrocarbon substance including an organic sulfide. The alkali borate and the organic sulfide can be allowed to interact across the borate solution-hydrocarbon substance interface to oxidize the organic sulfide. The oxidized organic sulfide is understood to accumulate in a polar borate solution. After a sufficient time, the hydrocarbon substance layer can have substantially no organic sulfide, or can have a concentration of organic sulfide greatly reduced from the concentration of organic sulfide in the hydrocarbon substance before combination with the alkali borate and solvent. A sufficient time for interaction can be determined, for example, by monitoring the concentration of the organic sulfide in the hydrocarbon substance, e.g., by monitoring the concentration of the organic sulfide through a gas chromatography-mass spectroscopy technique. The hydrocarbon substance layer can be separated from the borate solution layer, the borate solution layer including the oxidized organic sulfide. The separated, purified hydrocarbon substance can be used or further processed.

In one method, an alkali borate, e.g., sodium tetraborate or sodium perborate, is combined with a solvent, e.g., a polar solvent, and hydrogen peroxide to form a borate-hydrogen peroxide solution. The borate-hydrogen peroxide solution can be contacted, without being mixed, with a hydrocarbon substance including an organic sulfide. The alkali borate, hydrogen peroxide, and organic sulfide can interact across the interface between the borate hydrogen peroxide solution and the hydrocarbon substance to oxidize the organic sulfide. The oxidized organic sulfide is understood to accumulate in a polar borate-hydrogen peroxide solution. After a sufficient time, the hydrocarbon substance layer can have substantially no organic sulfide, or can have a concentration of organic sulfide greatly reduced from the concentration of organic sulfide in the hydrocarbon substance before combination with the alkali borate, hydrogen peroxide, and solvent. A sufficient time for interaction can be determined, for example, by monitoring the concentration of the organic sulfide in the hydrocarbon substance, e.g., by monitoring the concentration of the organic sulfide through a gas chromatography-mass spectroscopy technique. The hydrocarbon substance layer can be separated from the borate-hydrogen peroxide solution layer, the borate-hydrogen peroxide solution layer including the oxidized organic sulfide. The separated, purified hydrocarbon substance can be used or further processed.

### Example 1

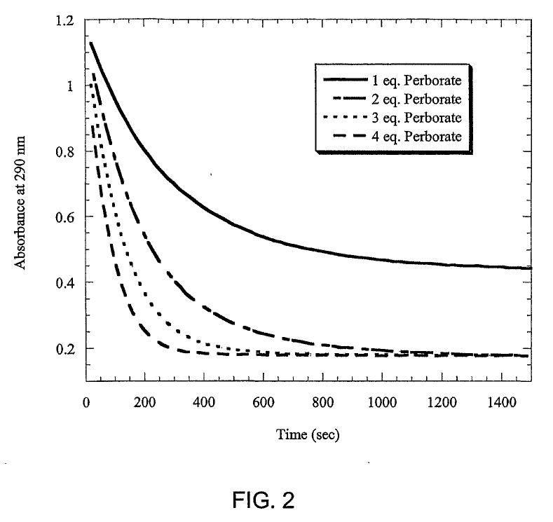

A 0.002 M solution of methyl phenyl sulfide in a solvent having acetonitrile and water in a 1:1 volumetric ratio (CH₃CN:H₂O (vol. 1:1)) was formed. 3.0 mL of the methyl phenyl sulfide solution was transferred to a quartz ultraviolet spectroscopy cell. An aliquot of 0.10 mL of a 0.06 M aqueous solution of sodium perborate tetrahydrate (NaBO₃.4H₂O) was formed and added to the 3.0 mL of the methyl phenyl sulfide solution in the ultraviolet cell and mixed at room temperature to form a solution in which the molar ratio of sodium perborate to methyl phenyl sulfide was 1:1 (1 eq. Perborate). Monitoring of the absorbance of 290 nm light by the contents of the spectroscopy cell commenced within 5 seconds of adding the aliquot and continued for at least 25 minutes; the absorbance of the 1 eq. perborate solution as a function of time is shown in FIG. 2. The absorbance of 290 nm light by methyl phenyl sulfone, the presumed product of oxidation of methyl phenyl sulfide, is minimal in comparison to the absorbance by methyl phenyl sulfide. The absorbance of 290 nm light by methyl phenyl sulfoxide is greater than the absorbance by methyl phenyl sulfone, but less than the absorbance by methyl phenyl sulfide. In a similar manner, aliquots of 0.20, 0.30, and 0.40 mL of 0.06 M aqueous solution of sodium perborate tetrahydrate were added to quartz ultraviolet spectroscopy cells containing 3.0 mL of the methyl phenyl sulfide solution and mixed to form solutions with sodium perborate and methyl phenyl sulfide in molar ratios of 2:1 (2 eq. Perborate), 3:1 (3 eq. Perborate), and 4:1 (4 eq. Perborate), respectively. Monitoring of the absorbance of 290 ran light by the contents of the spectroscopy cell commenced within 5 seconds of adding an aliquot and continued for at least 25 minutes; the absorbance as a function of time for each of the 2 eq., 3 eq., and 4 eq. perborate solutions is shown in FIG. 2.

To identify the product from the oxidization of methyl phenyl sulfide with sodium perborate, 2 mL of a 0.1 M solution of methyl phenyl sulfide in acetonitrile was added to 6.0 mL of CH₃CN:H₂O (vol. 1:1). 2 mL of a 0.2 M solution of aqueous sodium perborate was added and the resultant solution was stirred overnight at room temperature. A 1 mL aliquot from the reacted solution was added to solid potassium chloride to induce the separation of the solution into an aqueous layer and an acetonitrile layer. It is understood that the organic compounds, including any organic sulfide, sulfoxide, and sulfone compounds, were primarily present in the acetonitrile layer. Approximately 0.003 mL of the acetonitrile layer was subsequently injected into a Hewlett Packard 5890 Series II gas chromatograph (GC) coupled to a 5971A mass spectroscopy (MS) detector. The only compound detected in the aliquot was identified as methyl phenyl sulfone, based on comparison of the molecular weight and mass spectrum of the detected compound with the NIST Standard Reference Database (NIST98 and Search Program v. 1.7, Chem SW, Inc. version, 1999). This procedure was repeated for ethyl phenyl sulfide and diphenyl sulfide; quantitative conversion of ethyl phenyl sulfide and diphenyl sulfide to their respective sulfones was observed. Only 2 moles of sodium perborate per mole of methyl phenyl sulfide were necessary to oxidize the methyl phenyl sulfide to methyl phenyl sulfone.

### Example 2

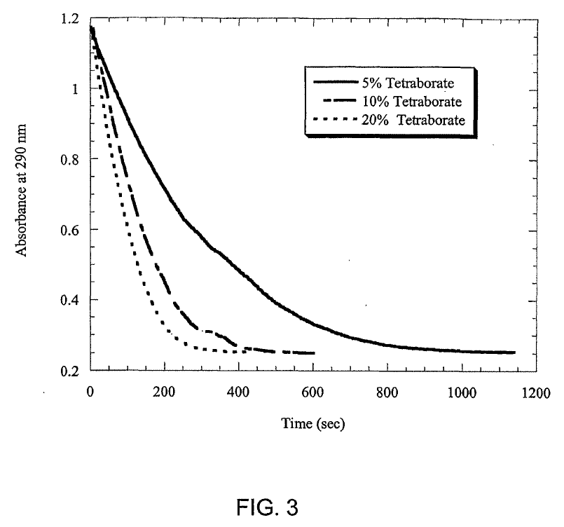

A solution having 0.002 M methyl phenyl sulfide and 0.08 M hydrogen peroxide in CH₃CN:H₂O (vol. 1:1) was added to a cuvette. An aliquot of 0.010 mL of 0.03 M aqueous sodium tetraborate decahydrate (Na₂B₄O₇.10H₂O) was added to 3.0 mL of the methyl phenyl sulfide-hydrogen peroxide solution at room temperature to form a solution having a molar ratio of sodium tetraborate to methyl phenyl sulfide of 5 mol % (5% Tetraborate). The change in the absorbance of 290 nm light by the methyl phenyl sulfide-hydrogen peroxide-sodium tetraborate solution in the cuvette was monitored. The absorbance of the solution in the cuvette reached its approximate minimum value in less than about 15 minutes. In a similar manner, aliquots of 0.020 and 0.040 mL of 0.03 M aqueous sodium tetraborate decahydrate were added to 3.0 mL volumes of the methyl phenyl sulfide-hydrogen peroxide solution at room temperature to form solutions having a molar ratio of sodium tetraborate to methyl phenyl sulfide of 10 mol % (10% Tetraborate) and 20 mol % (20% Tetraborate) respectively. The change in the absorbance of 290 nm light by the solutions of 5, 10, and 20 mol % sodium tetraborate was monitored and is presented in FIG. 3. For all three solutions, the absorbance of the solution in the cuvette reached its approximate minimum value in less than about 15 minutes. Hydrogen peroxide concentrations were determined by iodometric analysis. The results presented in FIG. 3 indicate that for a solution having 0.002 M methyl phenyl sulfide and 0.08 M hydrogen peroxide, the addition of only 5 mol % sodium tetraborate relative to methyl phenyl sulfide fully oxidized the methyl phenyl sulfide in less than about 15 minutes. It is understood that the full oxidization of the methyl phenyl sulfide with the presence of only a small amount of sodium tetraborate indicated that the sodium tetraborate functioned to catalyze reaction between the methyl phenyl sulfide and the hydrogen peroxide.

To determine the identity of the product resulting from the oxidization of methyl phenyl sulfide, 29.8 mg of sodium tetraborate decahydrate was added to 25 mL of a solution having 0.05 M of methyl phenyl sulfide and 2.0 M of hydrogen peroxide in CH₃CN:H₂O (vol. 1:1) to form a solution having a molar ratio of sodium tetraborate to methyl phenyl sulfide of 6.25 mol %. A 0.500 mL aliquot of the reacted solution was periodically removed from the reacting solution and added to solid potassium chloride. The potassium chloride induced the separation of the solution into an aqueous layer and an acetonitrile layer. Approximately 0.003 mL of the acetonitrile layer of the aliquot was subsequently injected into a Hewlett Packard 5890 Series II GC coupled to a 5971A MS detector. Compounds detected in an aliquot were identified with the NIST Standard Reference Database NIST98 and Search Program v. 1.7, Chem SW, Inc. version, 1999). The molar percentage of methyl phenyl sulfide, methyl phenyl sulfoxide, and methyl phenyl sulfone of the total methyl phenyl sulfide derivative product was determined for each aliquot removed from the reacting solution at a given time, as presented in Table A. The results presented in Table A indicate that for a solution having 0.05 M methyl phenyl sulfide and 2.0 M hydrogen peroxide, the addition of 6.25 mol % sodium tetraborate relative to methyl phenyl sulfide fully oxidized the methyl phenyl sulfide to methyl phenyl sulfone in about 260 minutes.

TABLE A

| Time (min) | Sulfide % | Sulfoxide % | Sulfone % |
|---|---|---|---|
| 10 | 65 | 35 | 0 |
| 20 | 43 | 57 | 0 |
| 30 | 14 | 86 | 0 |
| 40 | 12 | 88 | 0 |
| 50 | 3 | 97 | Trace |
| 60 | 2 | 98 | Trace |
| 70 | 0 | 97 | 3 |
| 80 | 0 | 98 | 2 |
| 90 | 0 | 96 | 4 |
| 100 | 0 | 81 | 19 |
| 110 | 0 | 82 | 18 |
| 120 | 0 | 65 | 35 |
| 130 | 0 | 54 | 46 |
| 140 | 0 | 53 | 47 |
| 150 | 0 | 45 | 55 |
| 160 | 0 | 30 | 70 |
| 170 | 0 | 32 | 68 |
| 180 | 0 | 29 | 71 |
| 190 | 0 | 17 | 83 |
| 200 | 0 | 17 | 83 |
| 210 | 0 | 9 | 91 |
| 220 | 0 | 5 | 95 |
| 230 | 0 | 7 | 93 |
| 240 | 0 | 5 | 95 |
| 260 | 0 | 0 | 100 |

### Example 3

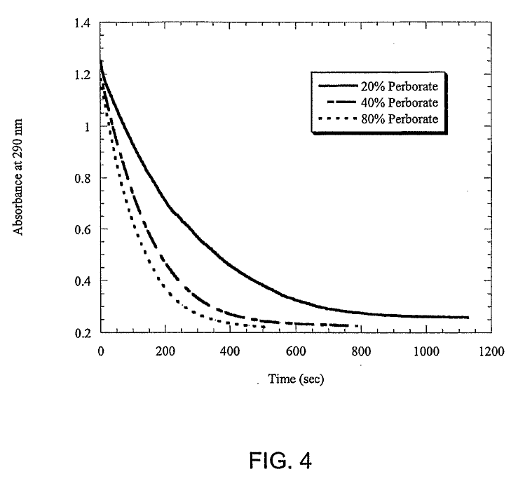

A solution having 0.002 M methyl phenyl sulfide and 0.08 M hydrogen peroxide in CH₃CN:H₂O (vol. 1:1) was added to a cuvette. An aliquot of 0.010 mL of 0.12 M aqueous sodium perborate tetrahydrate was added to 3.0 mL of the methyl phenyl sulfide-hydrogen peroxide solution at room temperature to form a solution having a molar ratio of sodium perborate to methyl phenyl sulfide of 20 mol % (20% Perborate). The change in the absorbance of 290 nm light by the methyl phenyl sulfide-hydrogen peroxide-sodium perborate solution in the cuvette was monitored. The absorbance of the solution in the cuvette reached its approximate minimum value in less than about 15 minutes. In a similar manner, aliquots of 0.020 and 0.040 mL of 0.12 M aqueous sodium perborate tetrahydrate were added to 3.0 mL volumes of the methyl phenyl sulfide-hydrogen peroxide solution at room temperature to form solutions having a molar ratio of sodium perborate to methyl phenyl sulfide of 40 mol % (40% Perborate) and 80 mol % (80% Perborate) respectively. The change in the absorbance of 290 nm light by the solutions of 20, 40, and 80 mol % sodium perborate was monitored and is presented in FIG. 4. For all three solutions, the absorbance of the solution in the cuvette reached its approximate minimum value in less than about 15 minutes. Hydrogen peroxide concentrations were determined by iodometric analysis. The results presented in FIG. 4 indicate that for a solution having 0.002 M methyl phenyl sulfide and 0.08 M hydrogen peroxide, the addition of only 20 mol % sodium perborate relative to methyl phenyl sulfide fully oxidized the methyl phenyl sulfide in less than about 15 minutes. It is understood that the full oxidization of the methyl phenyl sulfide with the presence of only a small amount of sodium perborate indicated that the sodium perborate functioned to catalyze reaction between the methyl phenyl sulfide and the hydrogen peroxide.

To determine the identity of the product resulting from oxidization of methyl phenyl sulfide, 48.1 mg of sodium perborate tetrahydrate was dissolved in 25 mL of a solution having 2.0 M of hydrogen peroxide in CH₃CN:H₂O (vol. 1:1). The reaction was initiated by adding 0.148 mL, that is, 1.25 mmol, of methyl phenyl sulfide, to form a solution having about 0.05 M of methyl phenyl sulfide and having a molar ratio of sodium perborate to methyl phenyl sulfide of 25 mol %. A 0.500 mL aliquot of the reacted solution was periodically removed from the reacting solution and added to solid potassium chloride. The potassium chloride induced the separation of the solution into an aqueous layer and an acetonitrile layer. A portion of the acetonitrile layer of the aliquot was subsequently injected into a Hewlett Packard 5890 Series II GC coupled to a 5971A MS detector. Compounds detected in an aliquot were identified with the NIST Standard Reference Database (NIST98 and Search Program v. 1.7, Chem SW, Inc. version, 1999). The molar percentage of methyl phenyl sulfide, methyl phenyl sulfoxide, and methyl phenyl sulfone of total methyl phenyl sulfide derivative product was determined for each aliquot removed from the reacting solution at a given time, as presented in Table B. The results presented in Table B indicate that for a solution having about 0.05 M methyl phenyl sulfide and 2.0 M hydrogen peroxide, the addition of 25 mol % sodium perborate relative to methyl phenyl sulfide fully oxidized the methyl phenyl sulfide to methyl phenyl sulfone in about 70 minutes.

TABLE B

| Time (min) | Sulfide % | Sulfoxide % | Sulfone % |
|---|---|---|---|
| 10 | 78 | 22 | 0 |
| 20 | 31 | 69 | 0 |
| 30 | 1.0 | 95 | 5.0 |
| 40 | 0 | 62 | 38 |
| 50 | 0 | 32 | 68 |
| 60 | 0 | 13 | 87 |
| 70 | 0 | 0 | 100 |

### Example 4

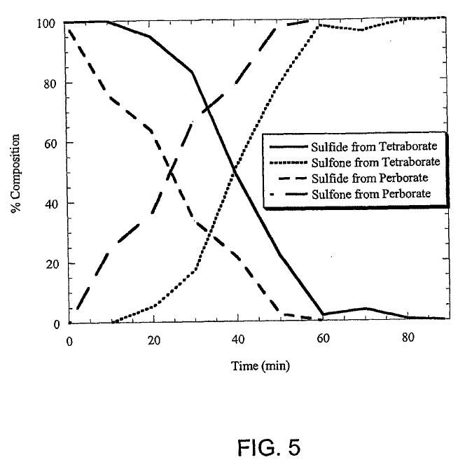

0.25 mmol of dibenzothiophene was dissolved in 20 mL of acetonitrile. 15 mL of a 0.67 M aqueous solution of hydrogen peroxide was added to the acetonitrile solution. 0.100 mL of a 0.0625 M aqueous solution of sodium tetraborate was added to the dibenzothiophene-hydrogen peroxide solution to form a resultant solution having a molar ratio of sodium tetraborate to dibenzothiophene of 2.5 mol %, and this resultant solution was allowed to react at room temperature. Iodometric analysis was utilized to determine the concentration of hydrogen peroxide. A 0.500 mL aliquot was removed from the reacting solution every 10 minutes and added to potassium chloride. The potassium chloride induced the separation of the solution into an aqueous layer and an acetonitrile layer. A portion of the acetonitrile layer of the aliquot was subsequently injected into a Hewlett Packard 5890 Series II GC coupled to a 5971A MS detector. Compounds detected in an aliquot were identified with the NIST Standard Reference Database (NIST98 and Search Program v. 1.7, Chem SW, Inc. version, 1999). No dibenzothiophene sulfoxide product was detected: the reaction appeared to convert dibenzothiophene (the sulfide) essentially directly to dibenzothiophene sulfone. The molar percentages of dibenzothiophene (the sulfide) and of dibenzothiophene sulfone of the total dibenzothiophene derivative product were determined for each aliquot removed from the reacting solution at a given time, as presented in Table C. The results presented in Table C indicate that for a solution of 0.25 mmol dibenzothiophene and 10 mmol of hydrogen peroxide in a solvent including about 4 volume parts acetonitrile to about 3 volume parts water, the addition of 2.5 mol % sodium tetraborate relative to dibenzothiophene oxidized the dibenzothiophene (the sulfide) to dibenzothiophene sulfone in about 90 minutes. The molar percentages of dibenzothiophene (the sulfide) and of dibenzothiophene sulfone of the total dibenzothiophene derivative product as a function of time are shown in FIG. 5 (Sulfide from Tetraborate and Sulfone from Tetraborate). It is understood that the oxidization of the dibenzothiophene with the presence of only a small amount of sodium tetraborate indicated that the sodium tetraborate functioned to catalyze reaction between the dibenzothiophene and the hydrogen peroxide.

TABLE C

| Time (min) | Sulfide | Sulfone |
|---|---|---|
| 10 | 100.00 | 0.0000 |
| 20 | 95 | 5 |
| 30 | 83 | 17 |
| 40 | 49 | 51 |
| 50 | 22 | 78 |
| 60 | 2 | 98 |
| 70 | 4 | 96 |
| 80 | 1 | 99 |
| 90 | 0 | 100 |

### Example 5

0.25 mmol of dibenzothiophene was dissolved in 20 mL of acetonitrile. 15 mL of a 0.67 M aqueous solution of hydrogen peroxide was added to the acetonitrile solution. 0.200 mL of a 0.125 M aqueous solution of sodium perborate was added to the dibenzothiophene-hydrogen peroxide solution to form a resultant solution having a molar ratio of sodium perborate to dibenzothiophene of 10 mol %, and this resultant solution was allowed to react at room temperature. Iodometric analysis was used to determine the concentration of hydrogen peroxide. A 0.500 mL aliquot was removed from the reacting solution every 10 minutes and added to potassium chloride. The potassium chloride induced the separation of the solution into an aqueous layer and an acetonitrile layer. A portion of the acetonitrile layer of the aliquot was subsequently injected into a Hewlett Packard 5890 Series II GC coupled to a 5971A MS detector. Compounds detected in an aliquot were identified with the NIST Standard Reference Database (NIST98 and Search Program v. 1.7, Chem SW, Inc. version, 1999). No dibenzothiophene sulfoxide product was detected: the reaction appeared to convert dibenzothiophene (the sulfide) essentially directly to dibenzothiophene sulfone. The molar percentages of dibenzothiophene (the sulfide) and of dibenzothiophene sulfone of the total dibenzothiophene derivative product were determined for each aliquot removed from the reacting solution at a given time, as presented in Table D. The results presented in Table D indicate that for a solution having 0.25 mmol dibenzothiophene and 10 mmol of hydrogen peroxide in a solvent including about 4 volume parts acetonitrile to about 3 volume parts water, the addition of 10 mol % sodium perborate relative to dibenzothiophene oxidized the dibenzothiophene (the sulfide) to dibenzothiophene sulfone in about 70 minutes. The molar percentages of dibenzothiophene (the sulfide) and of dibenzothiophene sulfone of the total dibenzothiophene derivative product as a function of time are shown in FIG. 5 (Sulfide from Perborate and Sulfone from Perborate). It is understood that the oxidization of the dibenzothiophene with the presence of only a small amount of sodium perborate indicated that the sodium perborate functioned to catalyze reaction between the dibenzothiophene and the hydrogen peroxide.

TABLE D

| Time (min) | Sulfide | Sulfone |
|---|---|---|
| 10 | 100 | 0 |
| 20 | 75 | 25 |
| 30 | 64 | 36 |
| 40 | 34 | 66 |
| 50 | 21 | 79 |
| 60 | 2 | 98 |
| 70 | 0 | 100 |

The embodiments illustrated and discussed in this specification are intended only to teach those skilled in the art the best way known to the inventors to make and use the invention. Nothing in this specification should be considered as limiting the scope of the present invention. All examples presented are representative and nonlimiting. The above described embodiments of the invention may be modified or varied, and elements added or omitted, without departing from the invention, as appreciated by those skilled in the art in light of the above teachings. It is therefore to be understood that, within the scope of the paragraphs and their equivalents, the invention may be practiced otherwise than as specifically described.

### REFERENCES

1. I. V. Babich and J. A. Moulijn, Fuel 82, 607-631 (2003); “Science and technology of novel processes for deep desulfurization of oil refinery streams: A review.”
2. S. K. Bej, S. K. Maity, U. T. Turaga, Energy & Fuels 18, 1227-1237 (2004); “Search for an Efficient 4,6-DMDBT Hydrodesulfurization Catalyst: A Review of Recent Studies.”
3. C. S. Song and X. L. Ma, Applied Catalysis B-Environmental 41, 207-238 (2003); “New design approaches to ultra-clean diesel fuels by deep desulfurization and deep dearomatization.”
4. C. S. Song, Catalysis Today 86, 211-263 (2003); “An overview of new approaches to deep desulfurization for ultra-clean gasoline, diesel fuel and jet fuel.”
5. Y. Okamoto, M. Breysse, G. M. Dhar, C. Song, Catal. Today 86, 1-3 (2003); “Effect of support in hydrotreating catalysis for ultra clean fuels.”
6. R. J. Angelici, Polyhedron 16, 3073-3088 (1997); “An overview of modeling studies in HDS, HDN and HDO catalysis.”
7. T. Aida, D. Yamamoto, M. Iwata, K. Sakata, Rev. Heteroatom Chem. 22, 241-256 (2000); “Development of oxidative desulfurization process for diesel fuel.”
8. A. McKillop and W. R. Sanderson, Tetrahedron 51, 6145-6166 (1995); “Sodium Perborate and Sodium Percarbonate—Cheap, Safe and Versatile Oxidizing-Agents For Organic-Synthesis.”
9. Kabe et al., Ind. Eng. Chem. Res. 31, 1577-1580 (1992); “Hydrodesulfurization of Sulfur-Containing Polyaromatic Compounds in Light Oil.”

What is claimed is:

1. A method for oxidizing an organic sulfide, comprising
combining an alkali borate, the organic sulfide, and a solvent; and
allowing the alkali borate and the organic sulfide to interact to produce an oxidized organic sulfide.

2. The method of claim 1, wherein the oxidized organic sulfide is an organic sulfone.

3. The method of claim 1, wherein the solvent is a polar solvent.

4. The method of claim 1, wherein the solvent comprises a polar compound selected from the group consisting of water, an alcohol, methanol, ethanol, 1-propanol, 2-propanol, 1-butanol, 2-butanol, 2-methyl-1-propanol, 2-methyl-2-propanol, acetone, N,N′-dimethylformamide, and acetonitrile, and combinations thereof.

5. The method of claim 1, wherein the solvent comprises acetonitrile.

6. The method of claim 1, wherein the solvent comprises a mixture of acetonitrile and water.

7. The method of claim 1, wherein the solvent comprises a mixture of acetonitrile and water having a volumetric ratio of from about 1:10 to about 10:1.

8. The method of claim 1, wherein the solvent comprises a mixture of acetonitrile and water having a volumetric ratio of from about 1:3 to about 3:1.

9. The method of claim 1, wherein the solvent comprises a mixture of acetonitrile and water having a volumetric ratio of about 1:1.

10. The method of claim 1, wherein the alkali borate is selected from the group consisting of lithium borate, sodium borate, and potassium borate.

11. The method of claim 1, wherein the alkali borate is a sodium borate.

12. The method of claim 1, wherein the alkali borate is an alkali tetraborate.

13. The method of claim 1, wherein the alkali borate is sodium tetraborate.

14. The method of claim 1, wherein the alkali borate is an alkali perborate.

15. The method of claim 1, wherein the alkali borate is sodium perborate.

16. The method of claim 1, further comprising
combining hydrogen peroxide with the alkali borate, the organic sulfide, and the solvent.

17. The method of claim 16,
wherein a molar ratio of the alkali borate to the organic sulfide is less than or equal to about 20 mol %.

18. The method of claim 16,
wherein a molar ratio of the alkali borate to the organic sulfide is less than or equal to about 5 mol %.

19. The method of claim 1, wherein the organic sulfide is selected from the group consisting of a mercaptane, an alkyl sulfide, an alkyl disulfide, an aryl sulfide, an aryl disulfide, a monoaromatic sulfur containing compound, thiophene, a polyaromatic sulfur containing compound, benzothiophene, and dibenzothiophene.

20. The method of claim 1, wherein the organic sulfide is selected from the group consisting of methyl phenyl sulfide, ethyl phenyl sulfide, diphenyl sulfide, and dibenzothiophene.

21. The method of claim 1, further comprising
combining hydrogen peroxide with the alkali borate, the organic sulfide, and the solvent,
wherein the organic sulfide is dibenzothiophene,
wherein the alkali borate is selected from the group consisting of sodium tetraborate and sodium perborate, and
wherein the solvent comprises acetonitrile and water.

22. The method of claim 1,
wherein the alkali borate is dissolved in the solvent to form a borate solution and
wherein the organic sulfide is in contact with, but not mixed with, the borate solution.

23. The method of claim 1, wherein the alkali borate, the organic sulfide, and the solvent are mixed.

24. The method of claim 1, wherein a hydrocarbon substance initially comprises the organic sulfide and the hydrocarbon substance is combined with the alkali borate and the solvent, further comprising separating the hydrocarbon substance from the alkali borate, the solvent, and the oxidized organic sulfide.

25. The method of claim 24, wherein the solvent is a polar solvent.

26. The method of claim 25,
wherein the hydrocarbon substance, the alkali borate, and the polar solvent are mixed to form a mixture and
wherein the mixture is allowed to separate into a polar layer, comprising the oxidized organic sulfide, and a nonpolar layer, comprising the hydrocarbon substance.

27. The method of claim 26, further comprising removing the nonpolar layer from the polar layer to obtain a purified hydrocarbon substance.

28. A composition for oxidizing an organic sulfide comprising an alkali borate, the organic sulfide, and a solvent.

29. The composition of claim 28, wherein the alkali borate is an alkali perborate and the solvent is a polar solvent.

30. The composition of claim 28, further comprising hydrogen peroxide.

31. The composition of claim 30, wherein the alkali borate is an alkali tetraborate and the solvent is a polar solvent.
